(19) United States
(12) Patent Application Publication
(10) Pub. No.: US 2017/0272429 A1
(43) Pub. Date: Sep. 21, 2017

(54) SYSTEM AND METHOD TO CONDUCT CLINICAL EXAMINATIONS AND SECURELY INTERACT WITH NETWORKED ELECTRONIC APPLICATIONS

(71) Applicant: Rajika Munasinghe, Troy, MI (US)

(72) Inventor: Rajika Munasinghe, Troy, MI (US)

(21) Appl. No.: 15/075,183

(22) Filed: Mar. 20, 2016

(51) Int. Cl. H04L 29/06; A61B 7/04; A61B 5/01; A61B 5/00; A61B 90/00; A61B 5/12; A61B 5/1455; G06F 17/30; A61B 90/30

(52) U.S. Cl. CPC H04L 63/0861; G06F 17/30879; A61B 7/04; A61B 5/01; A61B 90/30; A61B 90/361; A61B 5/123; A61B 5/14552; A61B 5/0053; A61B 2503/40; A61B 2560/0425; A61B 2560/0214

(57) ABSTRACT

Disclosed herein is a single portable computing device with components for secure authentication and subject identification with a set of integrated electronic diagnostic instruments that accompany a healthcare or veterinary professional as he or she examines or treats a subject before moving to another subject in another location. The device has a network interface so that secure access can be afforded to an electronic application for updating and observation. The disclosure permits clinicians to examine subjects and securely access protected information from an electronic medical record or similar veterinary application at different locations, that also reduces risk of transmitting infection, improves and facilities secure authentication and subject identification.

### TECHNICAL FIELD

This disclosure relates to a system and method for clinical evaluation of subjects in human and veterinary medicine and simultaneous interaction with networked electronic applications.

### BACKGROUND OF THE INVENTION

Clinical evaluation of human or veterinary subjects (collectively herein, “subject”) using diagnostic tools such as stethoscopes, flashlights, frequency generators, pulse oximeters, illuminated flexible scopes, vision and hearing testing is important in healthcare and veterinary practice. With recent advances in electronics, audio-visual equipment, microprocessor technology and networking, more sophisticated, portable diagnostic instruments capable of detecting and transmitting diagnostic information have been developed.

Healthcare and veterinary practice is increasingly dependent on physicians, nurses, patient care assistants, physical therapists, pharmacists, veterinarians, veterinary assistants and administrators (collectively herein, “provider”) using computers, electronic medical record systems, electronic health record systems, medical imaging systems, patient registries, administrative databases and clinical reference resources to provide care in the era of information technology. Computers that connect to large remote database servers and smaller local servers using secure networks are now the standard across the world. Healthcare and veterinary providers use a variety of devices to interact with these software applications, including but not limited to desktop computers, laptop computers, ultraportable computers, tablet devices and smartphones.

Using such mobile networked devices, interaction with healthcare and veterinary databases and software applications is being increasingly performed right at the point of care such as at the bedside or in procedure rooms in hospitals or surgical centers, at the examination rooms in ambulatory clinics and in patient homes by visiting physicians, nurses and other providers such as physical therapists and corresponding locations for veterinary practice.

In order to perform and document a detailed clinical assessment, providers need to carry the required diagnostic equipment as well a portable computer with them from location to location where clinical assessment and care is delivered. In doing so, contamination of the devices poses a risk from the standpoint of infection control. In addition to hand washing, non-destructive disinfection of electronic devices and diagnostic equipment is also necessary to reduce transmission of infections from one subject to another.

At each point of service, the provider needs to authenticate with the network or an application in order to securely review and document confidential protected health information.

In addition the provider needs to identify the correct subject that the examination is being performed using identifiers such as name, date of birth and medical record number.

When portable electronic diagnostic devices such as electronic stethoscopes and pulse oximeters and flashlights are used, a suitable power source is required for each device. Typically disposable or rechargeable power units are needed with each diagnostic tool requiring its own power unit. When the power unit is depleted, it needs to be replaced or recharged to permit continued use.

Publication (2009/0201636) discloses a portable, solvent resistant, ergonomic computing device that is easy to carry and disinfect for mobile healthcare applications. Publication (2012/0253851) discloses a system for controlling and displaying medical records on a display. That reference describes a means of displaying all or selected portions of the information displayed on the primary screen of the physician/clinician device (computer/tablet/phone) on to a secondary display that is connected to a network using an electronic network authentication protocol to improve the interaction between the clinician and the patient. Publication (7421367B2) discloses a handheld computing device with embedded environmental sensors.

### SUMMARY OF INVENTION

In the healthcare and veterinary environment of this disclosure's intended use, it would be desirable for the provider to use one integrated diagnostic and computing device to authenticate with a network, identify the correct subject, conduct clinical examinations and securely interact with electronic healthcare or veterinary applications thereby eliminating the need to carry a variety of different items.

It is desirable that automated authentication devices reduce the time and effort needed to access secure networks and electronic application. Using automated electronic identification systems reduce misidentification errors in documentation, clinician order entry, medication administration and in performing clinical procedures, thereby improving the safety of care provided to the subjects.

It would also be desirable for the different components of the device to share the same power source, visual display, network adapter, microphone, speakers, HDMI, USB, Power and headset connectors, eliminating the need to duplicate these components in each device. The single rechargeable power source can be swapped out and replaced replenishing power to all the components of the device immediately.

Further it would be also desirable to reduce the risk of infection transmission by disinfecting one device instead of multiple items. The disclosed device will be ergonomically designed with this specific purpose to limit the exposed surface area of the device and microbial resistant plastic that is easy to disinfect when the device is moved from one care location to another.

The disclosure offers a system and method for providers to authenticate using biometric and non biometric logon capability, identify subjects using automated electronic protocols, examine subjects using a several electronic diagnostic instruments and simultaneously access protected health information from an electronic medical record or comparable veterinary application at different locations with a single portable unit that reduces risk of transmitting infection.

### BRIEF DESCRIPTION OF THE DRAWINGS

FIG. 1 illustrates a first embodiment of the disclosed system;

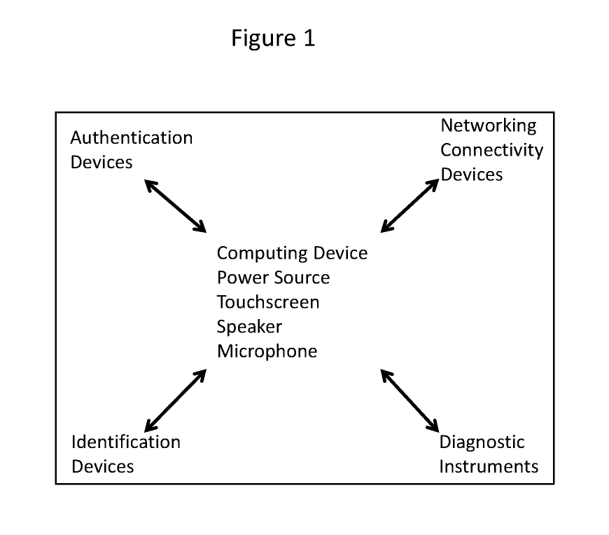

FIG. 2. illustrates networking and connectivity components

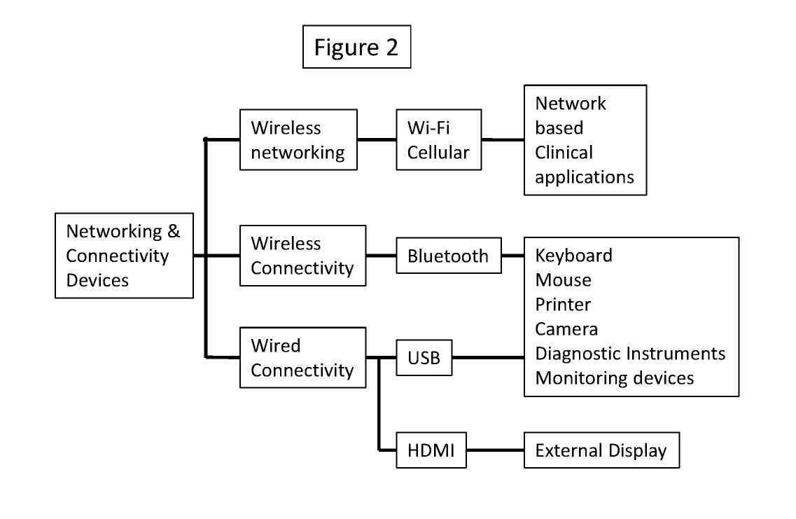

FIG. 3 illustrates a non-limiting list of integrated diagnostic instruments

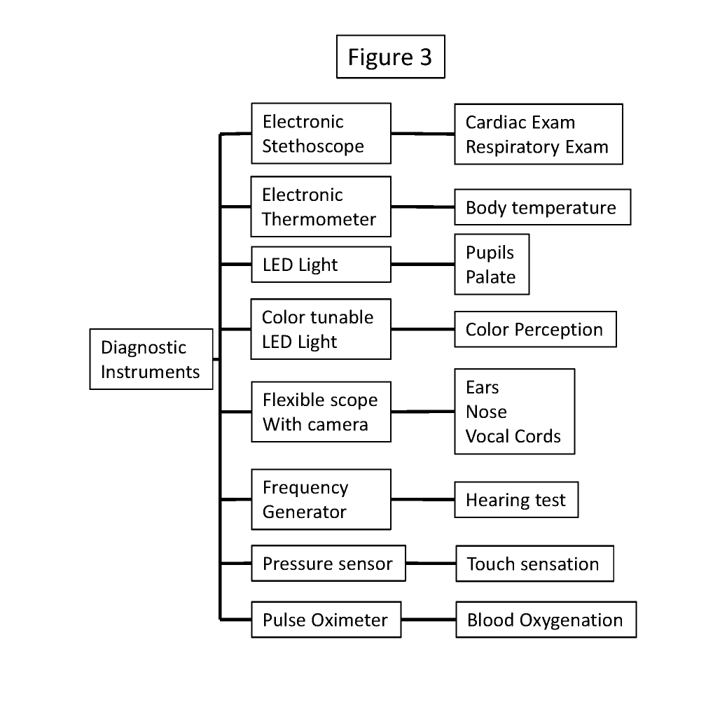

FIG. 4 depicts various optional process steps that need not be followed in the sequence shown.

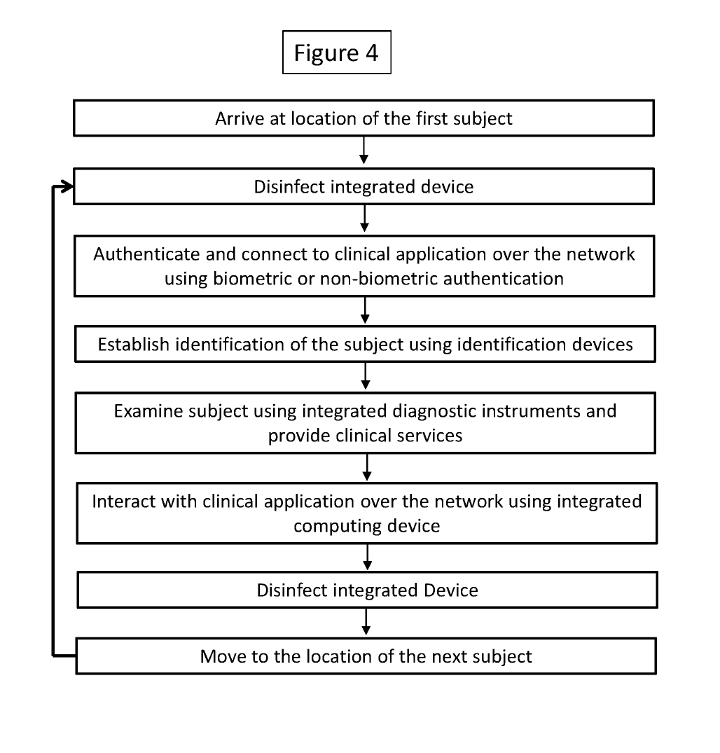

### DETAILED DESCRIPTION OF THE INVENTION

In one embodiment for use in the healthcare and veterinary industry is a system and method that incorporates components that include a networked computing device, biometric and non biometric authentication devices and protocols, electronic identifications devices and protocols, and a set of commonly used electronic diagnostic instruments in to one ergonomically designed unit housed within a single enclosure that utilizes the same power source, display, network adapter and controls.

This method allows the healthcare or veterinary care providers such as physicians, nurses, patient care assistants, physical therapists, pharmacists, veterinarians, veterinary assistants and administrators (collectively herein, “provider”) to move from one location to another and examine and provide care to subjects while interacting with a network-based, secure electronic medical record or other software application using a single device.

In one embodiment for use in the healthcare and veterinary industries, this device, optionally makes use of a thin client portable, low power computer unit, a rechargeable lithium ion battery or similar power source, a high definition touchscreen display, wireless network connectivity with Wi-Fi, cellular or comparable protocols, biometric logon devices such as a fingerprint scanner or retinal scanners, non biometric authentication devices such as barcode scanner, magnetic card readers or radio frequency identification devices and a set of diagnostic tools including but not limited to an electronic stethoscope for cardiac and respiratory examinations, an electronic digital thermometer, a flashlight to examine anatomical areas requiring illumination, LED with tunable color capability to check for color blindness, electronic frequency generator with volume control to check hearing perception in audiometric applications, an illuminated flexible scope with a high resolution miniature camera to examine anatomical areas not amenable to direct visualization, a tactile threshold and vibratory apparatus to examine sensory perception, an application for an electronic Snellen eye chart to check for visual acuity and a miniature electronic pulse oximeter to check blood oxygenation. In addition to an integrated high resolution touchscreen display unit, microphone and speakers, the device will have connections such as a HDMI or comparable connector to connect it to a larger display device such as a full size monitor or television and a connector such as a USB connector for the peripherals (keyboard, mouse and speakers) and wireless connectivity protocols such as Bluetooth and a power connector to charge the battery pack.

Optionally, the single computer device and the integrated diagnostic tools can be carried in a pocket, waist belt or holster. This frees up the hands for other tasks such as examining the subject, taking notes or providing other necessary aspects of care.

Any exposed components are designed to be small and can be thoroughly and easily disinfected. Sterilized components of the disclosed device and system minimize the risk of transmitting infectious agents from one subject to another.

Preferably disinfectant-resistant materials are used to permit easier disinfection of the exposed parts of the clinician's device.

In an alternate embodiment the system includes a disposable sack or disposable cover into which the device can be inserted before entering a zone of contamination. In use, the device stays in the covering medium. After use, the device is removed therefrom and the cover is discarded.

The device will have standard input output connectors such as USB and HDMI connectors to connect the device to a variety of peripherals such as a keyboard, pointing device, display device or other peripheral device such as card reader, memory card reader/writer, RFID, barcode scanner, camera, printer or clinical measurement instrument such as automated vital sign measurement device, or communication device. Wireless connectivity to such peripherals using protocols such as Bluetooth connectivity would be made available.

A variety of carrying systems is contemplated for the healthcare provider to wear the diagnostic and computer device including shoulder holsters, carrying belts (or holster) or custom designed pockets for lab coats and surgical scrubs.

A variety of peripherals can be custom configured with the device with connectors for different locations. Standard display monitors, keyboards and mice can be configured at desks and nursing stations. Wall mounted units can be configured out on hallways and outside patient rooms. Point of service configurations could include configurations that are ergonomically designed to help the provider interact with the patient, the device and the peripherals simultaneously in the most optimal way for the particular situation. For example, peripherals can be integrated with examination tables, hospital beds, transportation gurneys, bedside tables and other situations.

The inventor also contemplates developing several portable configurations that use small form factor keyboards, track pads and display goggles to connect with the device. This will enable providers to interact with smaller portable unit configurations under special circumstances such as providing point of service care in a patient room but use more easy to use traditional set of peripherals.

Using a multi-port peripheral connector such as a USB hub, the peripherals connecting with the device can be expanded to include devices such as magnetic card readers, memory card reader/writers, RFIDs, barcode scanners, external cameras, communication devices, printers, and devices such as automated thermometers, blood pressure meters and cardiac monitors.

When a provider arrive at the point of service, they would wipe down and disinfect the unit, authenticate with biometric or non biometric logon, identify the patient using an automated ID device, use the integrated diagnostic instruments to perform a clinical examination, interact with the electronic medical record using the built in computing device with its integrated touchscreen keyboard and display or connect the device it to a set of full size external peripherals situated a the point of care such as a keyboard, mouse and external monitor using the USB and HDMI connectors. The device will remain securely connected to a wireless network using a wireless protocol such as Wi-Fi or cellular network in order for the device to transmit and receive information from a server or cloud based electronic medical record or similar application during the period of interaction with the subject. Once care is completed, the provider will disconnect the device from any on site peripherals, then disinfect the device once again and move with the device to the next location and repeat the same steps over again.

One aspect of the disclosure includes design elements that reduce the size of the exposed parts to limit contamination and facilitate sterilization. The number of different items that needs to be sterilized or sanitized when moving between subjects is maintained at a minimum by integrating these components in to one portable unit within a single enclosure. Additionally integrating computing device with the multiple electronic diagnostic instruments to a single power source, network device, display, microphone, speakers, HDMI and USB connectors for wired connectivity to peripherals and wireless connectivity to peripherals using protocols such as Bluetooth will eliminate the need to have these capabilities in each individual component of the integrated unit.

This method, optionally makes use of a thin client portable computer unit such as the Raspberry Pi, Intel computestick, Lenovo ideacenter stick and a rechargeable lithium ion or similar power source, a high definition touchscreen display, wireless network connectivity with Wi-Fi, cellular or comparable protocols, biometric logon devices such as but not limited to fingerprint scanners, retinal scanners or voice recognition, non biometric authentication devices including but not limited to magnetic card readers and electronic chip readers, electronic identification and detection devices and protocols such as but not limited to barcode scanners, QR Codes, radio frequency identification devices and a set of diagnostic tools including but not limited to an electronic stethoscope such as Thinklabs one, an electronic thermometer such Braun Thermoscan, an LED flashlight or similar light source to examine pupils, palate and other anatomical areas requiring illumination, LED with tunable color capability or a color LED array to check for color blindness, electronic frequency generator with volume control to check hearing perception in audiometric applications, an illuminated flexible scope with a high resolution miniature camera to examine ears, throat, nose and other anatomical areas not amenable to direct visualization, a tactile threshold and vibratory apparatus such as the monofilament to check for sensory perception, an application for an electronic Snellen eye chart to check for visual acuity and a miniature electronic pulse oximeter such as the Veridian Deluxe Pulseoximeter. In addition to an integrated high resolution touchscreen display unit, microphone and speakers, the device will have connections such as a HDMI or comparable connector to connect it to a larger display device such as a full size monitor or television and a connector such as a USB connector for the peripherals (keyboard, mouse and speakers) and wireless connectivity protocols such as Bluetooth and a power connector to charge the battery pack.

Other features and advantages of the invention will become apparent to those skilled in the art upon review of the detailed description, claims and drawings in which like numerals are used to designate like features.

Various features of the invention are set forth in the following claims. It should be understood that the invention is not limited in its application to the details of construction and arrangements of the components set forth herein. The invention is capable of other embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

Certain terminology is used herein for purposes of reference only, and thus is not intended to be limiting. For example, terms such as “display device” refer to any computer system or display type for displaying received information. Terms such as program, interface, workstation are intended to provide contextual information for understanding the type of computing systems that may be used to implement the concepts described herein and are not intended to limit the present invention to specific hardware implementations. Such terminology may include the words specifically mentioned above, derivatives thereof, and words of similar import.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a server” and “a processor” can be understood to include one or more controllers or processors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network. It should be understood that a computer program may embrace constituent programs and that multiple programs may be implemented as a single or multiple programs.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications are hereby incorporated herein by reference in their entireties.

1. A system for a healthcare or veterinary provider to examine a subject and interact with a network based electronic medical record or comparable application comprising:
a portable, mobile, low power computing device and a rechargeable power source with touchscreen display, microphone, speaker, controls switches, wired and wireless connectivity to secure networks and external peripherals
an integrated electronic biometric and non-biometric authentication system using methods such as fingerprint scanners, retinal scanners, voice recognition, magnetic card readers and electronic chip readers
an integrated electronic subject identification system using methods such as barcode and QR scanners, magnetic card readers, chip readers and radio frequency identification
a set of integrated electronic diagnostic instruments such as but not limited to digital electronic stethoscope, an electronic thermometer, LED examination light with color tuning, flexible high resolution scope with camera, a frequency generator, a miniature pulse oximeter and a tactile pressure sensor.

2. The system of claim 1, wherein the single device is used to authenticate to a secure network or application, identify a human or veterinary subject for clinical evaluation, perform a clinical examination and securely interact with electronic applications using a network.

3. The system of claim 1, wherein the single device with networked computing and diagnostic instruments have fewer exposed components and surface area that can be thoroughly and easily disinfected so that the disinfected components may minimize the risk of transmitting infectious agents.

4. The system of claim 1, wherein the device can be carried in a pocket, waist belt or holster, thereby freeing up the hands for other tasks such as examining the subject, taking notes or providing other aspects of care.

5. The system of claim 1, further including a secure wireless network to which the computing device and diagnostic tools are connected using a secure wireless protocol such as Wi-Fi or a cellular network and a secure thin client utility.

6. The system of claim 5, further including a network with which the device is in secure communication as the provider moves from one location to another, while maintaining confidentiality of protected health information.

7. The system of claim 1, wherein the integrated device has a rechargeable battery that powers all the individual components and the computing device operates using an operating system.

8. The system of claim 1, utilizes the integrated biometric and non-biometric utilities to securely authenticate to the network and clinical applications to maintain integrity of protected health information.

9. The system of claim 1, uses automated electronic identifications methods and protocols to correctly identify the subjects being examined and receiving care by the provider.

10. A method for using the system of claim 1, comprising the step of:
disinfecting the integrated device prior to contact with the subject
authenticating to a secure network or application
verifying correct identification of the subject
performing clinical examination of the subject
securely observing and documenting confidential information on electronic applications.

11. The method of claim 9, further comprising the step of:
Using the HDMI, USB, DisplayPort, lightening or similar connectors or wireless adapters such as Bluetooth of similar to expand the capabilities of the system by connecting to a variety of external peripherals and instruments.

12. The method of claim 9, further including the step of:
using external peripherals and clinical instruments connected via the HDMI, USB or wireless adapters such as a stationary keyboard, a pointing device, a display device, and other USB based peripheral devices such as card reader, memory card reader/writer, RFID, barcode scanner, camera, printer, cardiac monitor and a clinical measurement instrument such as an automated vital sign measurement device and communication device.

13. The method of claim 9, further including the steps of:
disconnecting the device from external peripherals
disinfecting the device to reduce the risk of transmitting infection before moving to the location of the next subject.

14. The method of claim 9, further including the steps of:
deploying a disposable cover into which the device is inserted before the healthcare provider enters a zone of contamination
leaving the device in the cover while the device is in use; and after use, removing the device from the cover
discarding the cover when leaving the zone of contamination.

15. The system of claim 1, further including a carrying system associated with the healthcare provider and the device, the carrying system being selected from the group consisting of a shoulder holster, a belt clip or holster or a dedicated lab coat pocket.

16. The system of claim 1 has a common power source, a touchscreen display, audio components, HDMI and USB connectors, control switches and network interface for all of the integrated components.
